(19) United States
(12) Patent Application Publication
(10) Pub. No.: US 2025/0061992 A1
(43) Pub. Date: Feb. 20, 2025

(54) SYSTEM AND METHOD FOR ADAPTIVE GENERATION OF GRAPHICAL DATA OF A TREATMENT HISTORY

(71) Applicant: Roche Diabetes Care, Inc., Indianapolis, IN (US)

(72) Inventors: Samer Dajani, Carmel, IN (US); Chinmay Manohar, Carmel, IN (US)

(73) Assignee: Roche Diabetes Care, Inc., Indianapolis, IN (US)

(21) Appl. No.: 18/934,299

(22) Filed: Nov. 1, 2024

Related U.S. Application Data
(63) Continuation of application No. PCT/US2023/066473, filed on May 2, 2023.
(60) Provisional application No. 63/364,517, filed on May 11, 2022.

(51) Int. Cl. G16H 10/60; G06F 3/0482; G16H 40/20

(52) U.S. Cl. CPC G16H 10/60; G06F 3/0482; G16H 40/20

(57) ABSTRACT

A method for generating graphical data of a patient treatment history includes receiving medical data for the patient corresponding to patient visits to a healthcare provider and generating graphical data corresponding to a timeline view of the patient visits. The generated graphical data include first and second graphical elements corresponding to first and second diagnoses based on the medical data during a current patient visit and a prior patient visit. Each graphical element includes a graphical indicator of a diagnosis for a medical condition and at least one graphical sub-element, the at least one graphical sub-element being relevant to a physiological parameter selected from the medical data and related to the diagnosis. The method includes generating a first graphical connector between the second graphical element and the first graphical element, to indicate a progression of time between patient visits in the timeline view.

### CLAIM OF PRIORITY AND CROSS-REFERENCE TO RELATED APPLICATIONS

This application claims priority to Patent Cooperation Treaty Application No. PCT/US2023/066473, which is entitled “SYSTEM AND METHOD FOR ADAPTIVE GENERATION OF GRAPHICAL DATA OF A TREATMENT HISTORY,” and was filed on May 2, 2023, the entire contents of which are hereby incorporated herein by reference. This application claims further priority to U.S. Provisional Patent Application No. 63/364,517, which is entitled “SYSTEM AND METHOD FOR ADAPTIVE GENERATION OF GRAPHICAL DATA OF A TREATMENT HISTORY,” and was filed on May 11, 2022, the entire contents of which are hereby incorporated herein by reference. This application cross-references Patent Cooperation Treaty Application No. PCT/US2023/066475, which is entitled “SYSTEM AND METHOD FOR ADAPTIVE GENERATION OF GRAPHICAL DATA OF PREDICTED DIAGNOSES” and was filed on May 2, 2023, the entire contents of which are hereby incorporated herein by reference. This application further cross-references U.S. Provisional Patent Application No. 63/364,518, which is entitled “SYSTEM AND METHOD FOR ADAPTIVE GENERATION OF GRAPHICAL DATA OF PREDICTED DIAGNOSES” and was filed on May 11, 2022, the entire contents of which are hereby incorporated herein by reference.

### BACKGROUND

Persons with Diabetes (PwDs), and particularly those with type 2 diabetes (T2D), most often receive treatment from Primary Care Physicians (PCPs) such as General Practitioners (GPs) or Family Medicine Physicians (FMs). Primary care physicians are often overwhelmed due to high patient volumes, since they not only see patients with T2DM but also those with many other chronic conditions. With typical appointments lasting less than 15 minutes, there is a tremendous cognitive load on the PCPs when it comes to disease management and optimal therapy recommendations. These factors lead to the phenomenon of clinical inertia, which is the delay caused in appropriate intensification of therapies for better disease management. Clinical inertia, in turn, leads to high health-economic costs and negative quality of life implications for PwDs.

Therefore, there is a need for Clinical Decision Support (CDS) tools that aid PCPs and other healthcare providers (HCPs) in selecting the appropriate therapies for people with type 2 diabetes. When integrated within their clinical workflow, a CDS tool that takes into account patient characteristics can help HCPs in making better personalized therapy decisions improving clinical, patient-reported, and economical outcomes. While the medical field has recognized clinical guidelines for diabetes treatment from bodies such as the American Diabetes Association (ADA) and the European Association for the Study of Diabetes (EASD), even these clinical guidelines for therapy transitions in T2D can be cumbersome for a PCP to follow or apply given the volume of patient data. In addition, the potential courses of recommended therapy can be complex and evolve over several years, which introduce further difficulties in tracking of disease progress and the ability to provide proactive disease management. Consequently, improvements to systems and methods that provide clinical information and CDS to PCPs and other HCPs in an efficient manner that reduces cognitive load would be beneficial.

### SUMMARY

An adaptive clinical decision support user interface (UI) enables a health care provider (HCP) to provide efficient management of the care journey for a patient with diabetes. The UI contains information graphics for each visit that update dynamically with pertinent physiological values that are used to drive underlying guidance logic to form diagnoses and treatment recommendations. Comorbidities and related physiological markers can also be tracked to further adapt the care against accepted guidelines where a modified treatment may be recommended, advanced treatment services initiated, or disease risk progression algorithms called directly from the interface. The UI reduces the cognitive load in navigating complex therapeutic pathways based on the current disease state.

In one embodiment, a method for generating a user interface of a treatment history for a patient has been developed. The method includes receiving, with a processor, medical data for the patient, the medical data corresponding to a plurality of patient visits to a healthcare provider, and generating, with the processor, graphical data corresponding to a timeline view of the plurality of patient visits. The generating of the graphical data further includes generating a first graphical element corresponding to a first diagnosis based on the medical data during a current patient visit in the plurality of patient visits, the first graphical element further includes a graphical indicator of the first diagnosis for a first medical condition and at least one graphical sub-element, the at least one graphical sub-element being relevant to a physiological parameter selected from the medical data, the physiological parameter being related to the first diagnosis. The method further includes generating a second graphical element corresponding to a second diagnosis based on the medical data for the patient during a first prior patient visit in the plurality of patient visits, the second graphical element further includes a graphical indicator of the second diagnosis for a second medical condition and at least one graphical sub-element, the at least one graphical sub-element being relevant to a physiological parameter selected from the medical data, the physiological parameter being related to the second diagnosis. The method further includes generating a first graphical connector between the second graphical element and the first graphical element, the first graphical connector indicating a progression of time between the first prior patient visit and the current patient visit in the timeline view.

In another embodiment, a computing system configured to generate a user interface of a treatment history for a patient has been developed. The computing system includes a memory and a processor operatively connected to the memory. The memory is configured to store medical data for the patient, the medical data corresponding to a plurality of patient visits to a healthcare provider and stored program instructions. The processor is configured to execute the stored program instructions to generate graphical data corresponding to a timeline view. The processor is further configure to generate graphical data further including a first graphical element corresponding to a first diagnosis based on the medical data during a current patient visit in the plurality of patient visits, the first graphical element further includes a graphical indicator of the first diagnosis for a first medical condition and at least one graphical sub-element, the at least one graphical sub-element being relevant to a physiological parameter selected from the medical data, the physiological parameter being related to the first diagnosis. The processor is further configured to generate a second graphical element corresponding to a second diagnosis based on the medical data for the patient during a first prior patient visit in the plurality of patient visits, the second graphical element further includes a graphical indicator of the second diagnosis for a second medical condition and at least one graphical sub-element, the at least one graphical sub-element being relevant to a physiological parameter selected from the medical data, the physiological parameter being related to the second diagnosis. The processor is further configured to generate a first graphical connector between the second graphical element and the first graphical element, the first graphical connector indicating a progression of time between the first prior patient visit and the current patient visit in the timeline view.

In another embodiment, a method for generating a user interface of a treatment history for a patient has been developed. The method includes receiving, with a processor, medical data for the patient, the medical data corresponding to an initial patient visit to a healthcare provider and generating, with the processor, graphical data corresponding to a timeline view. The generating of the graphical data further includes generating a first graphical element corresponding to a first diagnosis based on the medical data during the initial patient visit, the first graphical element further includes a graphical indicator of the first diagnosis for a first medical condition and at least one graphical sub-element, the at least one graphical sub-element being relevant to a physiological parameter selected from the medical data, the physiological parameter being related to the first diagnosis.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 is a schematic diagram of a computer system that is configured to generate graphical data of a medical timeline for a patient in an adaptive user interface for a healthcare provider.

FIG. 2 is a block diagram of a method of operation for the system of FIG. 1.

FIG. 3 is a depiction of a timeline view that incorporates graphical data of a treatment history for a patient.

FIG. 4 is a depiction of the timeline view of FIG. 3 depicting a larger time range in the timeline view.

FIG. 5 is a depiction of the timeline view of FIG. 3 shifted to depict an earlier time range in the history of patient visits in the timeline view.

FIG. 6 is a depiction of a simplified timeline view during an initial patient visit.

### DETAILED DESCRIPTION

These and other advantages, effects, features and objects are better understood from the following description. In the description, reference is made to the accompanying drawings, which form a part hereof and in which there is shown by way of illustration, not limitation, embodiments of the inventive concept. Corresponding reference numbers indicate corresponding parts throughout the several views of the drawings.

While the inventive concept is susceptible to various modifications and alternative forms, exemplary embodiments thereof are shown by way of example in the drawings and are herein described in detail. It should be understood, however, that the description of exemplary embodiments that follows is not intended to limit the inventive concept to the particular forms disclosed, but on the contrary, the intention is to cover all advantages, effects, and features falling within the spirit and scope thereof as defined by the embodiments described herein and the embodiments below. Reference should therefore be made to the embodiments described herein and embodiments below for interpreting the scope of the inventive concept. As such, it should be noted that the embodiments described herein may have advantages, effects, and features useful in solving other problems.

The devices, systems and methods now will be described more fully hereinafter with reference to the accompanying drawings, in which some, but not all embodiments of the inventive concept are shown. Indeed, the devices, systems and methods may be embodied in many different forms and should not be construed as limited to the embodiments set forth herein; rather, these embodiments are provided so that this disclosure will satisfy applicable legal requirements.

Likewise, many modifications and other embodiments of the devices, systems and methods described herein will come to mind to one of skill in the art to which the disclosure pertains having the benefit of the teachings presented in the foregoing descriptions and the associated drawings. Therefore, it is to be understood that the devices, systems and methods are not to be limited to the specific embodiments disclosed and that modifications and other embodiments are intended to be included within the scope of the embodiments. Although specific terms are employed herein, they are used in a generic and descriptive sense only and not for purposes of limitation.

Unless defined otherwise, all technical and scientific terms used herein have the same meaning as commonly understood by one of skill in the art to which the disclosure pertains. Although any methods and materials similar to or equivalent to those described herein can be used in the practice or testing of the methods, the preferred methods and materials are described herein.

Moreover, reference to an element by the indefinite article “a” or “an” does not exclude the possibility that more than one element is present, unless the context clearly requires that there be one and only one element. The indefinite article “a” or “an” thus usually means “at least one.” Likewise, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. For example, the expressions “A has B,” “A comprises B” and “A includes B” may refer both to a situation in which, besides B, no other element is present in A (i.e., a situation in which A solely and exclusively consists of B) or to a situation in which, besides B, one or more further elements are present in A, such as element C, elements C and D, or even further elements.

As used herein, the term Person with Diabetes (PwD) refers to a patient who is diagnosed with or is at-risk for being diagnosed with one or more forms of diabetes including of pre-diabetes, type 1 diabetes, type 2 diabetes, gestational diabetes, as well as one or more comorbidities that are associated with diabetes. In the specific embodiments described herein, the PwD is a patient of a healthcare provider (HCP), and references to the PwD and a patient are used interchangeably herein. While the specific embodiments that are described herein are directed to improving the user interface of the patient treatment history for HCPs who treat PwDs, the systems and methods described herein are not limited to the treatment of PwDs and may be used to implement improved user interfaces for the treatment of other diseases and medical conditions, and particularly chronic medical conditions that require long term treatment.

As used herein, the term “physiological parameter” refers to any quantifiable aspect of a PwD's physiology that is measured as part of providing medical data to diagnose a new medical condition or to track the state of a previously diagnosed medical condition. A non-limiting list of physiological parameters that are of interest to the treatment of diabetes and diabetes comorbidities includes body mass index (BMI), blood pressure (BP), blood glucose, glycosylated hemoglobin (HbAlc), blood ketones, and estimated glomerular filtration rate (cGFR).

As used herein, the term “medical data” refers to include both medical diagnostic data and medical treatment data. The medical diagnostic data include identifications of prior diagnoses, diagnostic test results, and records of prior and current physiological parameter values for the PwD. The medical diagnostic data optionally include relevant genetic data, phenotype data, demographic data, and socio-economic data pertaining to the PwD. The medical treatment data include records of previously prescribed medications or other medical treatments that have been prescribed to the PwD during previous patient visits. During a current patient visit, a clinical decision support system is configured to generate one or more prescribed treatments for a PwD based on the medical data and an HCP can adopt a prescribed treatment or manually select a different course of action for the PwD.

As used herein, the term “prescribed treatment” refers to any medical diagnostic tests, medical diagnostic or prognostic algorithms, medical procedures, medical therapies, medications, diet and lifestyle modification, or other recommended course of action that the HCP issues to the PwD during the treatment history for the PwD. In particular, regarding medications the term “prescribed” here encompasses both over-the-counter and prescription medications.

As used herein, the term “graphics data” refers to any form of encoded data that a computing device uses to generate a visually perceptible output including text, geometry, pictures, icons, textures, and the like using a display device, printer, or other output device. Different forms of graphics data include both static image data and moving images such as animations and video. Examples of graphics data include rasterized image data, vector graphics data, procedural graphics data, and combinations thereof. Examples of rasterized image data include graphics data that encode an array of pixel values in an image, where a display device generates an output image formed from the array of pixel values. Rasterized image data may be compressed using the JPEG, PNG, WEBP, or other suitable compression formats for static images and using video compression codecs such as h.264, h.265, VP9, AV1, or other suitable compression formats for video or animations. Examples of vector graphics include graphics data that encode declarative parameters that describe the shapes, colors, arrangements, and other details of an image that a computing device processes to reproduce an image, and examples of vector graphics include the scalable vector graphics (SVG), graphics generated from cascading style sheet (CSS) documents, portable document format (PDF), and other suitable vector graphics formats. Procedural graphics data includes data encoded as imperative command data that a processor executes to generate graphics data in a dynamic manner. Examples of procedural graphics data include encoded command parameters to control JavaScript, WebAssembly, WebGL, or another scripting language to draw graphics as part of the HTML <canvas> element used in publicly available web browsers, or data encoded in the Postscript language that a computing device renders using a Postscript rendering engine. Furthermore, markup language formats such as the hypertext markup language (HTML), extensible markup language (XML), or the suitable markup languages may be used to format the arrangement of one or more sets of graphics data that form graphical elements to generate the timeline views and other graphics described herein. In some configurations, a single computing system generates graphics data and performs the process of rendering the graphics data to a display device for human users to view the graphics. As described in further detail below, in other configurations a first computing system generates the graphics data and transmits the graphics data to one or more computing systems that perform the task of rendering the graphics to one or more display devices to enable one or more human users to view the graphics.

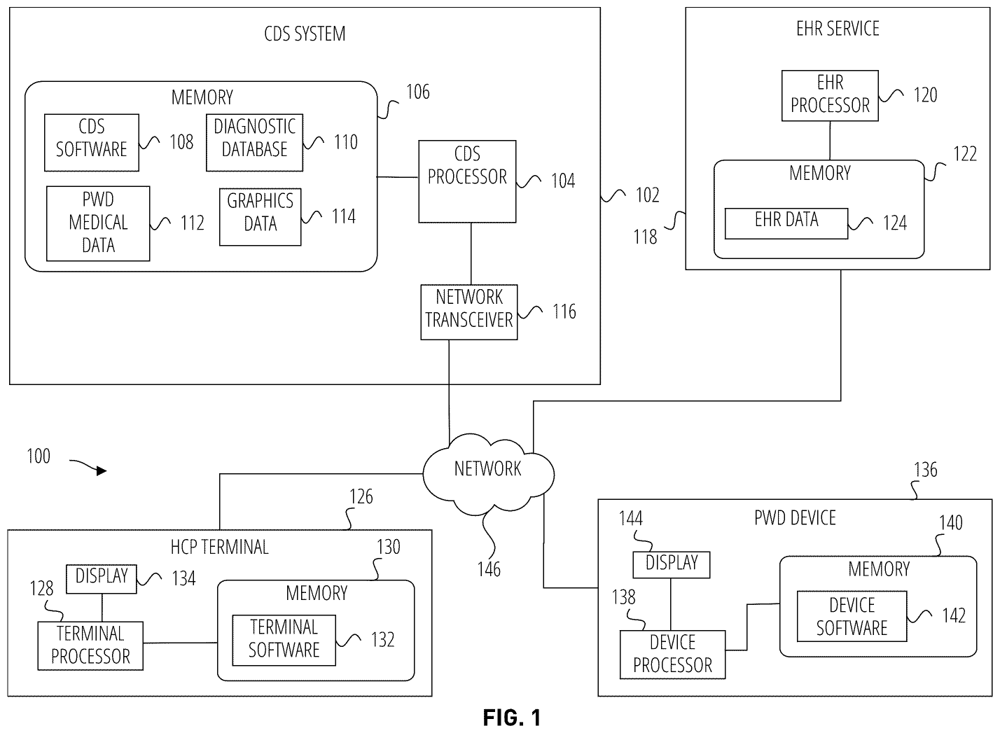

FIG. 1 depicts a system 100 that provides clinical decision support information to HCPs with the adaptive user interfaces described herein. The system 100 includes a clinical decision support (CDS) system 102, an electronic health record (EHR) service 118, an HCP terminal 126, and an optional PwD device 136. The CDS system 102, electronic health record (EHR) SERVICE 118, HCP terminal 126, and PWD device 136 are communicatively connected via a network 146.

The CDS system 102 of FIG. 1 performs the functions described herein utilizing one or more computing devices that include one or more central processing units (CPUs), graphics processing units (GPUs), application specific integrated circuits (ASICs), field programmable gate arrays (FPGAs), other digital logic devices, or combinations thereof that are depicted as the CDS processor 104 in FIG. 1. The CDS processor 104 is operatively connected to a CDS memory 106 and a network transceiver 116. The CDS memory 106 includes one or more volatile data storage devices such as static and dynamic random access memory (RAM) and one or more non-volatile data storage devices such as magnetic, solid state, and optical storage drives. During operation, the CDS processor 104 reads and writes data to the CDS memory 106 to execute stored program instructions and to store data including medical data received from the HCP terminal 126 and the EHR service 118 and generated graphics data. The CDS processor 104 operates the network transceiver 116, which is a wired or wireless network interface controller that transmits and receives data via the network 146, to receive medical data and other commands from the HCP terminal 126, transmit and receive EHR data with the EHR service 118, and transmit graphical data of a timeline view user interface of treatment history for the PwD to the HCP terminal 126. In some configurations, the CDS processor 104 also operates the network transceiver 116 to receive medical data directly from the PWD device 136 and to transmit the graphical data of a treatment history for the PwD to the PWD device 136.

In the CDS system 102, the CDS memory 106 stores CDS SOFTWARE 108, a diagnostic database 110, PwD medical data 112, and graphics data 114. The CDS SOFTWARE 108 includes stored program instructions that the CDS processor 104 executes to perform the clinical support functions and generate graphical data corresponding to a timeline view of one or more diagnoses for the PwD and one or more prescribed treatments to treat the PwD over a series of patient visits. The CDS SOFTWARE 108 also implements one or more network interfaces, such as web servers or other network servers, to enable the HCP terminal 126 to access and send commands to the CDS system 102 and to enable the CDS system 102 to transmit the generated graphical data described herein to the HCP terminal 126. The diagnostic database 110 includes a stored set of logical rules that the CDS system 102 uses to generate diagnoses for the PwD based on the medical data for the PwD. In one specific configuration, the diagnostic database 110 encodes guidelines from the American Diabetic Association (ADA) Standards of Medical Care in Diabetes. Alternative configurations employ different medical guidelines or other algorithms to generate diagnoses and prescribed treatments. While the CDS system 102 uses the diagnostic database 110 to generate graphical elements that display diagnoses and prescribed treatments in an automated manner during operation of the system 100, an HCP optionally uses the HCP terminal 126 to override the suggested diagnosis or course of action recommendations. The PwD medical data 112 include any relevant medical data for the PwD during both a current patient visit and historic medical record data including medical data for one or more prior patient visits. The PwD medical data 112 optionally include PwD medical data that the CDS system 102 receives from the EHR service 118 with historic medical data stored in an electronic health record for the PwD as well as data received from external lab tests, home diagnostics such as spot and continuous blood glucose meter devices, and medical data that an HCP provides to the CDS system 102 via the HCP terminal 126 during a patient visit. Furthermore, during operation the CDS system 102 optionally transmits updated medical data for the PwD to the EHR service 118 to reflect updated measurements to physiological parameters, diagnoses, or to record prescribed medications or other medical treatments that the PwD receives during a course of treatment. The graphics data 114 include a graphical representation of a timeline view user interface of a treatment history for the PwD over a series of one or more patient visits based on the PWD medical data 112. For example, the graphics data 114 form a timeline view including graphical elements and sub-elements to depict one or more diagnoses and relevant physiological parameters for each diagnosis, prescribed treatments, prescribed medications, diagnostic tests, and graphical connectors that link the graphical elements in the timeline view. In some configurations, the graphics data 114 further include stored text, icons, geometric templates, and other visually perceivable data that the CDS system 102 uses for adaptive generation of the timeline view for the PwD based on the PwD medical data 112.

The EHR service 118 of FIG. 1 provides medical data for the PwD to the CDS system 102 in the form of an EHR. In the illustrative embodiment of FIG. 1, the EHR service is a networked computing service that includes a digital processor 120, a memory 122 that stores the EHR data 124 for the PwD. The EHR data 124 is a digital record that is encoded in a standard format such as the Fast Healthcare Interoperability Resource (FHIR) format, a version of the Health Level Seven International (HL7) format, or another suitable electronic health record format. In the embodiment of FIG. 1, the EHR service 118 operates independently of the CDS system 102, but in an alternative configuration the EHR service 118 and the CDS system 102 may be implemented as a unified system. In practical embodiments, the EHR data 124 for the PwD may include data received from multiple data sources including external diagnostic test services, the HCP who operates the HCP terminal 126, the CDS system 102, and EHR data from other HCPs such as medical specialists who treat the PwD for other medical conditions and comorbidities.

The HCP terminal 126 of FIG. 1 is a desktop or laptop personal computer (PC), tablet, smartphone, or other suitable client computing device of the HCP that includes a terminal processor 128, memory 130, and a display device 134. The HCP uses the HCP terminal 126 to provide medical data to the CDS system 102 and optionally the EHR services 118, and to receive and display graphical data corresponding to a timeline view of a plurality of patient visits for a PwD as the PwD receives treatment over a series of visits with the HCP. In some configurations, the HCP also uses the HCP terminal 126 to communicate with the PWD device 136 between patient visits or to conduct remote patient visits in situations where the HCP provides telehealth services to the PwD. In the embodiment of FIG. 1, HCP terminal 126 executes stored program instructions in the terminal software 132 stored in the HCP terminal memory 130 to enable the HCP terminal 126 to communicate with the CDS service 102. In one configuration, the terminal software 132 includes operating system and web browser software that acts as a client to one or more web services provided by the CDS system 102, but in alternative configurations the terminal software 132 is another client software program. During operation, the terminal processor 128 executes the terminal software 132 and operates the display device 134 to generate a visible output of the graphics data 114 that the CDS system 102 generates and transmits to the HCP terminal 126. The display device 134 is, for example, a flat panel display screen or other electronic display device, although in some configurations a printer may reproduce the graphical display of the timeline view on paper or another print medium. In some configurations, the display device 134 incorporates a touchscreen interface to enable the HCP to enter data and modify the timeline view as described in further detail below, although in other configurations the HCP terminal 126 incorporates a combination of a mouse, keyboard, voice input device, or other input devices [not shown] to receive HCP input.

The PWD device 136 of FIG. 1 is another desktop or laptop PC, tablet, smartphone, or other suitable client computing device of the PwD that includes a device processor 138, a device memory 140 that stores PwD device software 142, and a device display 144. In some configurations, the PWD device 136 receives physiological parameter data from a monitoring device such as a spot or continuous blood glucose meter, a fitness tracking device such as a smart watch, or other medical device. The PWD device 136 is optionally configured to enable the PwD to conduct a telehealth patient visit with the HCP via the HCP terminal 126 using videoconferencing software and other telehealth software that is otherwise known to the art and is not described in further detail herein. In some configurations, the CDS system 102 transmits generated graphics data for the timeline view user interface of the treatment history for the PwD to the PWD device 136 for display using the display device 144 via the network 146. In another configuration, the HCP terminal 126 re-transmits the graphics data for the timeline view of the treatment history for the PwD to the PWD device 136 during a telehealth patient visit.

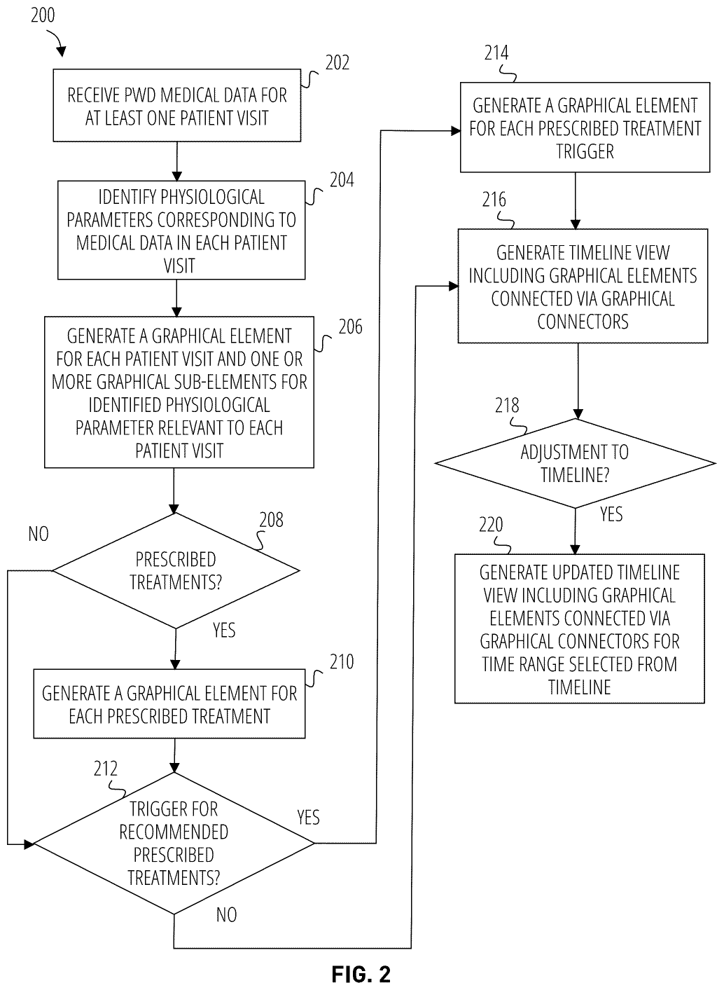

FIG. 2 is a block diagram of a process 200 for the operation of a CDS system to generate a graphical timeline view user interface of a treatment history of diagnosed conditions and recommended prescribed therapies for a PwD over a series of one or more patient visits. While the process 200 can be performed at any time, for illustrative purposes the process 200 is described as occurring during a patient visit to generate the graphical timeline view of the current patient visit and of one or more prior patient visits if such prior patient visits have occurred. The process 200 is described in conjunction with the system 100 of FIG. 1, and a reference to the process 200 performing a function or action refers to the operation of a processor, such as the CDS processor 104 in CDS system 102, to execute stored program instructions, such as the CDS SOFTWARE 108, to perform the function or action.

The process 200 begins as the CDS system 102 receives PwD medical data for at least one patient visit (block 202). In the system 100, processor 104 in the CDS system 102 receives the medical data from one or more sources including, but not limited to, the EHR service 118, the HCP terminal 126, and, in some instances, the PWD device 136. In particular, the EHR service 118 provides medical data for prior patient visits including prior diagnoses, prescribed medications and medical treatments, a historic record of physiological parameter data measurements for the PwD, and optionally socio-economic and demographic data pertaining to the PwD. During a patient visit, the HCP terminal 126 optionally transmits physiological parameter and other medical data to the CDS system 102 based on manual input from the HCP, from automatically uploaded physiological parameters generated by medical testing devices such as blood glucose meters, or both. Additionally, the CDS system 102 may receive medical data regarding blood tests or other diagnostic tests that the PwD receives at an external diagnostic laboratory prior to the patient visit either directly from a computing system of the diagnostic laboratory, via the EHR service 118, or from the HCP terminal 126. As described above, the CDS system 102 stores the received PwD medical data 112 in the CDS memory 106.

The process 200 continues as the CDS system 102 identifies physiological parameters that have the greatest relevance to a diagnosis during each patient visit (block 204). In the embodiment of FIG. 1, the CDS processor 104 uses the diagnostic database 110 to identify the physiological parameters in the PwD medical data 112 that have the greatest relevance to each diagnosis in a series of one or more patient visits. For example, if a patient visit includes diagnoses that a PwD is both obese and has uncontrolled HbAlc, which indicate the onset of type 2 diabetes, then the CDS processor 104 uses the diagnostic database 110 to identify the physiological parameters that have the highest relevance to these diagnoses, such as body mass index (BMI) related to obesity and the measured HbAlc level for the PwD. In many instances, the PwD medical data 112 include physiological parameters and other information that are not directly relevant to a diagnosis, and the CDS processor 104 filters these data from the timeline view, although an HCP may of course access the full PwD medical data 112 via a traditional user interface if desired.

The process 200 continues as the CDS system 102 generates a graphical element for each patient visit and at least one graphical sub-element for identified physiological parameters that are relevant to one or more diagnoses in each patient visit (block 206). Graphical elements and graphical sub-elements provide graphical indicators of the physiological parameter data that led to the diagnosis and prescribed treatment. A graphical indicator refers to any type of graphics data in the timeline view that conveys specific information about the medical data for the PwD, a prescribed treatment for the PwD, or a recommendation for a prescribed treatment for the PwD. A graphical sub-element is a type of graphical element that is subordinate to another graphical element in the timeline view, and the CDS system 102 generates the graphics data for the graphical sub-elements within the borders of a parent graphical element or otherwise associates each graphical sub-element with a parent graphical element. The graphical elements and sub-elements provide a clear indication of the relationship between a graphical element for a patient visit and one or more graphical sub-elements that are related to the patient visit.

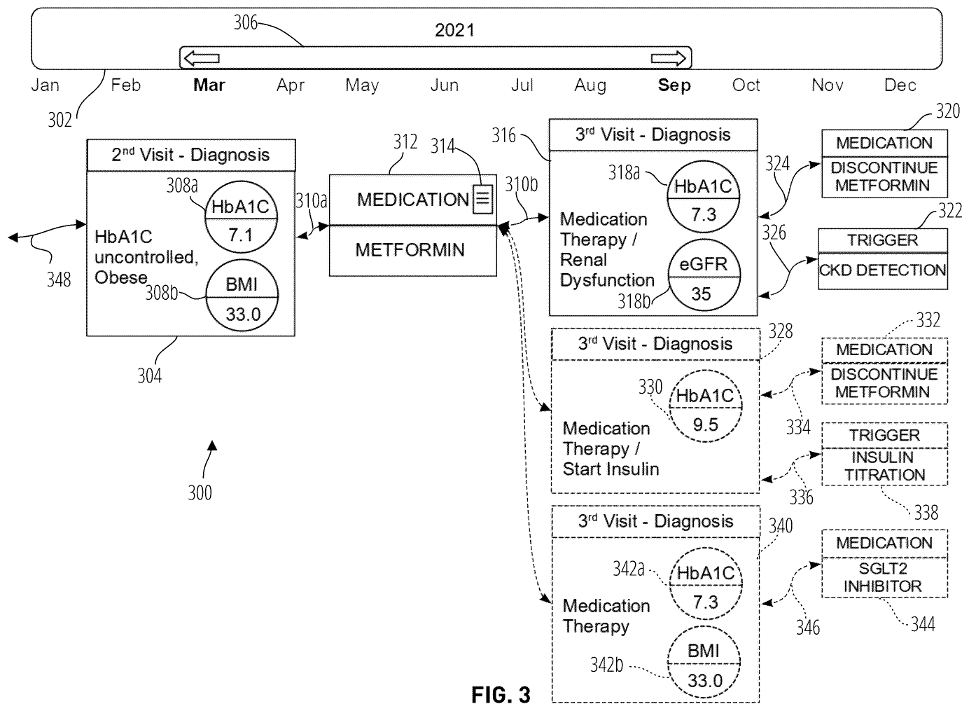

FIG. 3 depicts one example of a timeline view 300 that is rendered from the graphics data that are generated during the process 200 including graphical elements and graphical sub-elements for each patient visit. In the timeline view 300, the graphical elements 304 and 316 are embodied as rectangular graphical elements that each include text to provide information relevant to each patient visit including graphical indicators of the number of the patient visit (e.g. first, second, third, etc.), the current diagnoses, and the current medications or therapies prescribed to the PwD, if any. FIG. 3 depicts one example of the timeline view the graphical element 304 includes text graphical indicators that indicate the second PwD visit and that the PwD is diagnosed as being obese with uncontrolled HbAlc. The graphical sub-elements 308a and 308b are depicted as circles that are sub-divided into two semicircular regions with a top region that lists the physiological parameter that is related to the diagnoses and treatments and a bottom region that lists a quantified value of the physiological parameter. In FIG. 3, the sub-element 308a depicts HbAlc with a value of 7.1, which is an elevated value supports a diagnosis prediction that the patient has diabetes. The sub-element 308b depicts a body mass index (BMI) physiological parameter that is relevant to a diagnosis of obesity. In some configurations, the graphical data of each physiological parameter graphical sub-element provide additional information beyond the quantitative value of the physiological parameters to the HCP. For example, in some configurations the physiological parameter data are displayed using color-coded text or color-coded graphics in a graphical sub-element to indicate if the physiological parameters are in-range or out-of-range for a particular PwD. Using the HbAlc physiological parameter as an example, a green color can indicate an HbAlc value that is considered normal for a healthy individual, a yellow color can indicate an elevated HbAlc for a non-insulin dependent diabetic, and a red color can indicates an even more elevated HbAlc that indicates a need for insulin therapy. In some configurations, the graphical sub-element of a physiological parameter also includes an arrow or other graphical indicator that displays a trend of the physiological parameter over time from the prior patient visit, such as an up or down arrow, which indicates a rising or falling trend the HbAlc level or changes in another relevant physiological parameter. In some configurations, the graphical indicators of physiological parameters include graphs, icons, or other non-text graphical indicators that an HCP can easily interpret to assess the condition of the PwD.

The timeline view 300 further depicts the second patient visit and a third patient visit graphical element 316 that is spatially offset from the graphical element 304 in the timeline view and the two elements are linked by a first graphical connector 310a/310b that indicates a progression of time between patient visits along the timeline view. In FIG. 3, the graphical element 316 for the third patient visit represents the current patient visit while the second patient visit in the graphical element 304 represents the most recent previous patient visit, and in some embodiments the default timeline view depicts the current patient visit and the most recent prior patient visit where applicable. For a treatment history that includes one or more prior patient visits that are not displayed in the default timeline view, the timeline view 300 optionally includes a graphical connector 348 extending from the earliest displayed patient visit, which is graphical element 304 in the example of FIG. 3, to provide a graphical indication that additional earlier patient visits are present in the treatment history. The graphical element 316 further includes a graphical indication that the PwD is on a medication therapy and a diagnosis for renal dysfunction, which is a known comorbidity of diabetes. The graphical element 316 further includes graphical sub-elements 318a and 318b. The graphical sub-element 318a indicates the HbAlc value for the patient at the time of the third patient visit, which is the same physiological parameter as the sub-element 308a but with a potentially different value (7.3 vs. 7.1) based on changes in the patient's physiology. The graphical sub-element 318b indicates an estimated Glomerular Filtration Rate (cGFR) value that is a physiological parameter of high relevance to the renal dysfunction diagnosis. Thus, the graphical element 316 includes a graphical sub-element for at least one physiological parameter (cGFR) that is not present in graphical element 304 for the earlier patient visit, and conversely the graphical element 304 includes a graphical sub-element for at least one physiological parameter (BMI) that is not present in the graphical element 310.

As described above, the process 200 identifies and generates the graphical sub-elements for the physiological parameters of greatest relevance to the diagnosis in each patient visit, and in some instances the selected physiological parameters of greatest relevance change based on changes in the underlying condition of the PwD. As such, the process 200 generates an adaptive display of information that emphasizes the physiological parameters of highest relevance during different patient visits in the medical history of the PwD, which enables the HCP to assess and treat the PwD efficiently and with reduced cognitive load compared to reviewing all of the physiological parameters for the PwD in a traditional EHR or other health record. For example, FIG. 3 further depicts non-limiting examples of alternative graphical elements 328 and 338 that the CDS system 102 generates during the process 200 based on different potential diagnoses and relevant physiological parameters for the PwD that could occur for different disease progressions. The graphical element 328 corresponds to a diagnosis that the PwD should start insulin because the HbAlc value depicted in graphical sub-element 330 has increased substantially from the previous patient visit and an existing prescription of metformin is no longer effective. The graphical element 340 corresponds to a diagnosis that the PwD should continue to receive non-insulin medications in response to a small increase in HbAlc (graphical sub-element 342a) and an unchanged BMI (graphical sub-element 342b). FIG. 3 depicts the graphical elements 328 and 340 with the corresponding graphical sub-elements in dashed lines because in this example only a single displayed graphical element 316 corresponds to the assessed condition of the PwD during the patient visit. In some embodiments, an HCP can modify the diagnosis for the current patient visit via, for example, a drop-down list box or other user interface element, to manually select a different diagnosis and set of treatment options for the PwD. The CDS system 102 updates graphical sub-elements as needed to generate graphical data for the most relevant physiological parameters related to the selected diagnosis.

Referring again to FIG. 2, during the process 200 the CDS processor 104 identifies prescribed treatments, if any, for the PwD in the PwD medical data 112 (block 208) and generates a graphical element for each prescribed treatment in the timeline view (block 210). In the processing of blocks 208 and 210, the prescribed treatments refer to previously prescribed treatments that are recorded in the PwD medical data 112 as part of the medical history for the PwD. Because a prescribed treatment is typically issued during a patient visit and subsequently provided to the PwD after the patient visit, the CDS processor 104 generates the timeline view with the graphical elements of prescribed treatments located at an intermediate position between the graphical elements for the patient visits with the graphical connector that links the patient visits being further subdivided into two sub-connectors that also depict the prescribed treatment in the timeline view.

Referring to FIG. 3, the graphical element 312 depicts a previously prescribed treatment, such as a medication prescription for metformin, including a label that the prescribed treatment is a medication and an identifier for the type of medication. In alternative embodiments, the graphical element for a prescribed medication depicts additional details such as the dosage level and regimen or other pertinent information about the medication. The first graphical connector includes a first sub-connector element 310a that links the graphical element 304 for the prior patient visit to the graphical element 312 of the prescribed treatment and the second sub-connector element and 310b that connects the graphical element 312 of the prescribed treatment to the graphical element 316 of the current patient visit to provide a clear indication of when the prescribed treatment occurred in the timeline view 300.

As described herein, during the process 200 an HCP optionally elects to prescribe a treatment that deviates from one or more recommended prescribed treatments that the CDS system 102 provides in the timeline view 300. If the HCP issues another prescribed treatment, the CDS system 102 stores a data identifier in association with the manually selected prescribed treatment to identify that the prescribed treatment was selected manually in the PwD medical data 112. When the CDS system 102 generates an updated timeline view 300 that includes the manually selected prescribed treatment at a later time, the CDS processor 104 generates the graphical element with a specific graphical indicator identifying that the HCP selected the prescribed treatment manually instead of from one of the recommended prescribed treatments. For example, the CDS processor 104 generates the graphical element of a prescribed treatment using a specific color or graphical icon to identify that the HCP selected the prescribed treatment manually, while the graphical elements for prescribed treatments that conform to the recommendations from the CDS system 102 use a different color or other graphical indicators.

During a patient visit, the HCP optionally records clinical notes related to the diagnosis and prescribed treatment. In the embodiment of FIG. 3, the graphical element 312 includes a graphical sub-element for a clinical note 314, which is depicted as an icon that the HCP may select to display the previously recorded clinical notes for a patient visit. The clinical notes include any data that the HCP records related to the patient visit and prescribed medical treatment, such as the rationale for prescribing the treatment, reminders for a follow up patient visit. During operation, the HCP optionally selects the graphical sub-element for the clinical note 314 to produce a tooltip or pop-up window that displays the text of the clinical notes for review. The HCP can close the tooltip or pop-up window to return to the display of the timeline view 300 to ensure that the HCP can utilize the timeline view 300 with minimal distraction.

Referring again to FIG. 2, the process 200 continues as the CDS processor 104 identifies triggers for recommended prescribed treatments if any, for the PwD based on the physiological data for in the PwD medical data 112 for the current patient visit and the criteria in the diagnostic database 110 (block 212). If one or more prescribed treatments are identified, the CDS processor 104 generates a graphical element for a trigger of each recommended prescribed treatment in the timeline view (block 214). Each trigger graphical element includes a graphical indicator of a recommendation for a prescribed treatment that is relevant to a diagnosis in the first patient visit. In some configurations, more than one trigger is generated if multiple prescribed treatments are applicable to the PwD. The CDS processor 104 also generates a graphical connector between the graphical element for the current patient visit and a corresponding graphical element for the trigger. The trigger graphical elements for the recommended prescribed treatments enable the HCP to select a prescribed treatment in an efficient manner, although the HCP may decline to select one or more of the triggers, or may elect to prescribe a different treatment than the recommendations that the CDS system 102 generates based on the PwD medical data 112 and the diagnostic database 110. If the HCP selects one or more of the triggers for recommended prescribed treatments, then the CDS system 102 stores a record of the selected prescribed treatments in the PwD medical data 112 and optionally transmits the prescribed treatment data to the EHR service 118 to maintain a record of the prescribed treatments.

Referring to FIG. 3, the timeline view 300 includes trigger graphical elements 320 and 322 that correspond to the current patient visit graphical element 316. The graphical element 320 includes a trigger for a change in an existing medication treatment, which in this case is a recommendation to discontinue an existing metformin regimen. In particular, to identify a trigger for discontinuing a therapy the CDS processor 104 identifies that the therapy is in effect based on the PwD medical data 112 and that discontinuing the therapy is a recommended treatment option based on the diagnosis, which is for renal dysfunction in the example of FIG. 3, using the diagnostic database 110. The graphical element 322 is a trigger for a chronic kidney disease (CKD) detection algorithm or clinical test that provides the HCP with further information about the diagnosed renal dysfunction. In the timeline view 300, graphical connectors 324 and 326 link the patient visit graphical element 316 to the trigger graphical elements 320 and 322, respectively. During operation, the HCP uses the HCP terminal 126 to select a hyperlink or other graphical user interface control element on a trigger element to select the trigger element. For example, the HCP selects a hyperlink in the graphical element 320 to activate a separate user interface (not shown) for the PwD's prescribed medications to discontinue the metformin prescription. Similarly, the HCP selects a hyperlink in the graphical element 322 to open a web site or other user interface to order the CKD detection algorithm or clinical test for the PwD. The HCP optionally enters clinical notes for the patient visit after selecting one or more of the triggers or selecting a prescribed treatment that differs from the recommended prescribed treatment triggers.

As described above, the CDS system 102 generates the graphical data in the timeline view 300 in an adaptive manner based on different potential diagnoses for the PwD. In FIG. 3, the trigger graphical elements 332 and 338 represent discontinuing a metformin regimen and triggering an insulin titration protocol, respectively, for a PwD who has a recommendation to begin insulin therapy based on the diagnosis of graphical element 328. Graphical connectors 334 and 336 connect the patient visit graphical element 328 to the trigger graphical elements 332 and 338, respectively. Similarly, the trigger element 344 indicates a recommendation for an SGLT 2 inhibitor prescribed treatment, and a graphical connector 346 connects the patient visit graphical element 340 to the trigger graphical element 344. As described above with reference to the graphical elements 328 and 340, the CDS processor 104 only generates the graphical elements 332 and 338 if the diagnosis matches the diagnosis of uncontrolled HbAlc indicating insulin dependency in the graphical element 328, and the CDS processor 104 only generates the graphical element 344 if the diagnosis matches the diagnosis of elevated HbAlc that can be controlled by non-insulin medications in the graphical element 338.

Referring again to FIG. 2, the process 200 continues as the CDS processor 104 generates the timeline view that includes the graphical elements and connectors that are described above with reference to the processing of blocks 206-214 (block 216). In addition to including an arrangement of the previously generated graphical elements, the timeline view generates one or more graphical element that depict a time range over which the patient visits and prescribed therapies occur. The timeline view further includes a timeline slider that enables an HCP to adjust both the size of the time range that is depicted in the timeline view and to move the time range depicted in the timeline view forward and backward in time.

With reference to FIG. 3, the timeline graphical element 302 includes a display of dates during which the displayed graphical elements 304 and 316 for the patient visits occurred. In the specific example of FIG. 3, the timeline view 302 depicts the year 2021 in a rectangular region with the abbreviate month names (Jan., Feb., Mar., . . . ) of 2021 arranged linearly in the timeline. The timeline view 300 aligns the graphical elements of the patient visits with the dates of each visit, with the graphical element 304 corresponding to a patient visit in March of 2021 and the graphical element 316 corresponding to a visit in September of 2021. The timeline graphical element 302 also highlights the entries for March and September in bold font to emphasize the date of each visit. While FIG. 3 depicts the level of precision for the date of each patient visit with a year and month for PwDs who visit the HCP a small number of times each year. For patients that require more frequent patient visits, the timeline graphical element presents more precise date information including the day and optionally time-of-day for each patient visit.

Upon generation of the timeline view in block 216 of the process 200, the CDS system 102 uses the CDS processor 104 and network transceiver 116 to transmit the generated graphical data for the timeline view user interface to the client HCP terminal 126 via the network 146. The HCP terminal 126 receives the graphical data and the terminal processor 128 executes the terminal software 132, such as a web browser or other client software, to generate a rendered user interface with a visual depiction of the timeline view using the display device 134 that is provided in the HCP terminal 126. The HCP interacts with the user interface using the HCP terminal 126, and as described below the HCP optionally provides input to the HCP terminal to update the timeline user interface, which the CDS system 102 receives via the network 146 and processes to provide an updated set of graphical data for an updated timeline view to the HCP terminal 126. Additionally, in some configurations the CDS system 102 optionally transmits the generated graphical data for the timeline view user interface to the PWD device 136 for direct display to the PwD using the display device 144. While the system 100 is embodied as a networked system in which the CDS system 102 is connected to the HCP terminal 126 via the network 146 for illustrative purposes, in another configuration a single computing system performs the operations of both the CDS system 102 and the HCP terminal 126. In this configuration, a processor in a single computing system generates the graphical data corresponding to the timeline view of patient visits and operates a display device that is provided in the single computing system to display the graphical data corresponding to the timeline view of the patient visits. In one configuration, the single computing system is the HCP terminal 126 that is further reconfigured to host the CDS software 108, diagnostic database 110, PwD data 112, and graphics data 114 in addition to the terminal software 132.

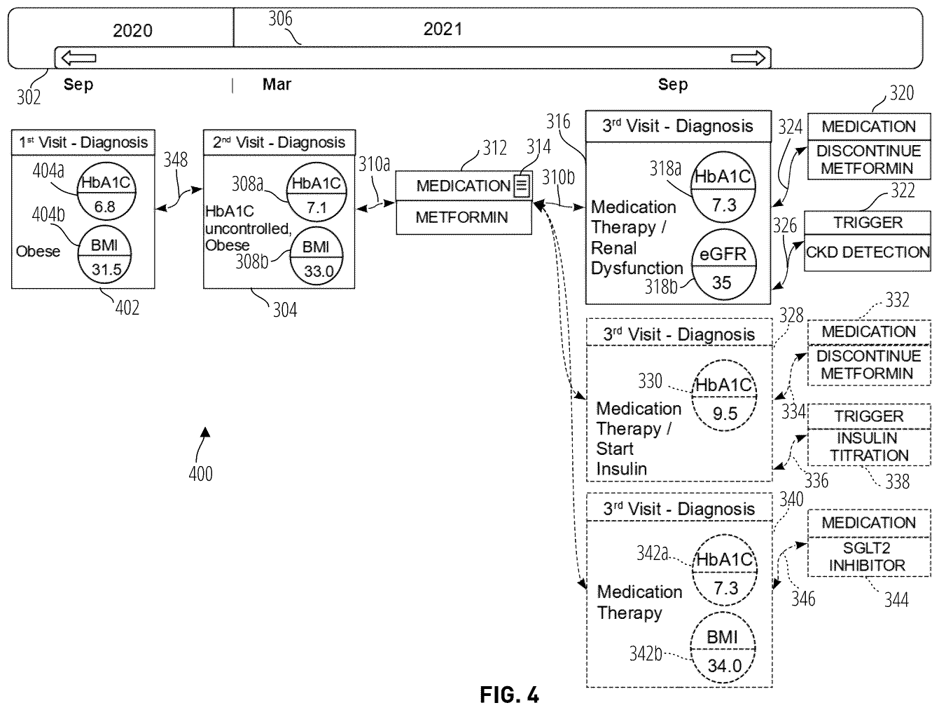

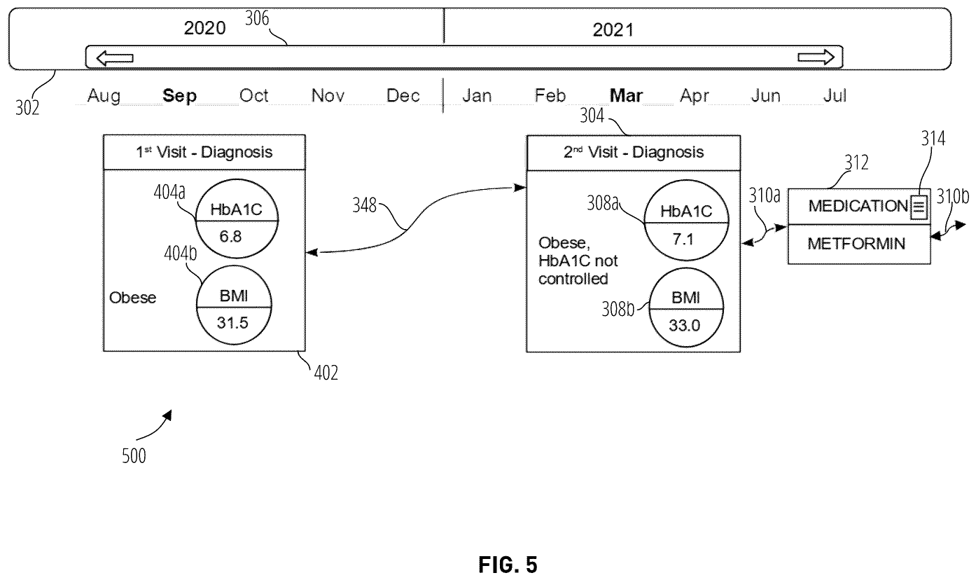

Referring to FIG. 2 and FIG. 3, the HCP optionally adjusts the timeline view using a timeline slider to increase or decrease the time range in the timeline view 300 or to move the time range in the timeline view 300 (block 218). If the CDS system 102 receives an input from the HCP terminal 126 to adjust the timeline view, then the CDS system 102 generates graphics data for an updated timeline view that depicts the time range specified in the input from the HCP terminal 126 (block 220). As depicted in FIG. 3, the timeline graphical element 302 further includes a timeline slider 306. The timeline slider 306 covers the range of time that is presented in the timeline view 300, which enables an HCP to adjust the timeline view 300 by providing input via the HCP terminal 126. In one mode of operation, the timeline slider 306 receives an input to adjust the size of the timeline slider to increase or decrease the size of the time range that is depicted in the timeline view 300. For example, in one operation the HCP clicks or drags one of end arrows depicted in the timeline slider 306 to increase or decrease the size of the time range depicted in the timeline view, and FIG. 4 depicts the results of this operation in more detail. In another operation, the HCP performs a click-and-drag operation to move the timeline slider 306 to move the time range that is depicted in the timeline view without increasing or decreasing the size of the time range, and FIG. 5 depicts the results of this operation in more detail. In one configuration, an HCP adjusts the size of the time range in the timeline view or moves the time range of the view based on calendar dates, while in another configuration the time increment for each adjustment is based on the series of patient visits stored in the PwD medical data 112. In a configuration that uses patient visits as a basis for adjusting the timeline view, changing the size of the time range depicted in the timeline view adds or removes one or more patient visits to the timeline view instead of adding or removing a specific number of months or years to the timeline view, while moving the timeline view updates the display of patient visits by one or more patient visits forward or backwards in time instead of moving the timeline view by a specific number of months or years.

FIG. 4 depicts an adjusted timeline view 400 that includes a larger time range in response to input from the HCP that expands the time range to be depicted in the timeline view during the process 200. In particular, the timeline view 400 includes graphical elements from the timeline view 300 of FIG. 3 and further includes a graphical element 402 corresponding to a first patient visit for the PwD. Similarly to the graphical elements 304 and 316, the graphical element 402 includes graphical depiction of a diagnosis for the first patient visit (“obese”) and two graphical sub-elements 404a and 404b that depict HbAlc (404a) and BMI (404b). The graphical connector 348 between the graphical elements 402 and 304 indicates a progression of time from the first patient visit depicted in graphical element 402 to the second patient visit depicted in graphical element 304. The CDS processor 104 generates graphics data corresponding to the first patient visit in the graphical element 402, graphical sub-elements 404a and 404b, and the graphical connector 348 based on the PwD medical data 112 and the diagnostic database 110 in the same manner described above with reference to the processing of blocks 206-210. Additionally, the CDS processor 104 updates the timeline graphical element 302 to depict the adjusted time range, which in the example of FIG. 4 includes September 2020 to September of 2021. In the example of FIG. 4, the timeline graphical element 302 includes identifiers for the years 2020 and 2021 and further includes a reduced-detail listing of only the months when a patient visit occurred to provide the HCP with relevant date information for each patient visit. While FIG. 4 depicts an operation that increases the time range depicted in the timeline view 400, those of skill in the art will appreciate that a similar operation can decrease the time range that is depicted in the timeline view as well.

FIG. 5 depicts an adjusted timeline view 500 that the CDS processor 104 generates in response to input from the HCP to move the timeline view to an earlier time range in the treatment history for the PwD. The timeline view 500 includes graphical elements and graphical sub-elements for the first and second patient visits that are depicted above, including the graphical element 402 and graphical sub-elements 404a and 404b for the first patient visit and graphical element 304 and graphical sub-elements 308a and 308b for the second patient visit. The graphical connector 348 indicates a progression of time between the first and second patient visits and the timeline view 302 depicts the adjusted timeline overlapping portions of 2020 and 2021 with highlighted months indicating the date of each patient visit. The timeline view 500 also includes the graphical element 312 corresponding to the prescribed treatment from the second medical visit and the graphical sub-connectors 310a and 310b that indicate an additional treatment history beyond the second patient visit. To generate the timeline view 500, the CDS processor 104 removes the graphical element 316 corresponding to the current patient visit from the timeline view because the current patient visit occurs outside of the earlier time range in the timeline view 500.

Those of skill in the art will recognize that the processing of steps 206-216 may occur in a different order than what is described above or concurrently. Additionally, the timeline views depicted herein are arranged in a left-to-right format representing earlier to more recent times, but alternative configurations can orient the timeline in right-to-left format or vertically in a top-to-bottom or bottom-to-top format. Additionally, the alternative configurations of the process 200 can include different visual formats and arrangements of the graphical elements, graphical sub-elements, and graphical connectors that are depicted herein.

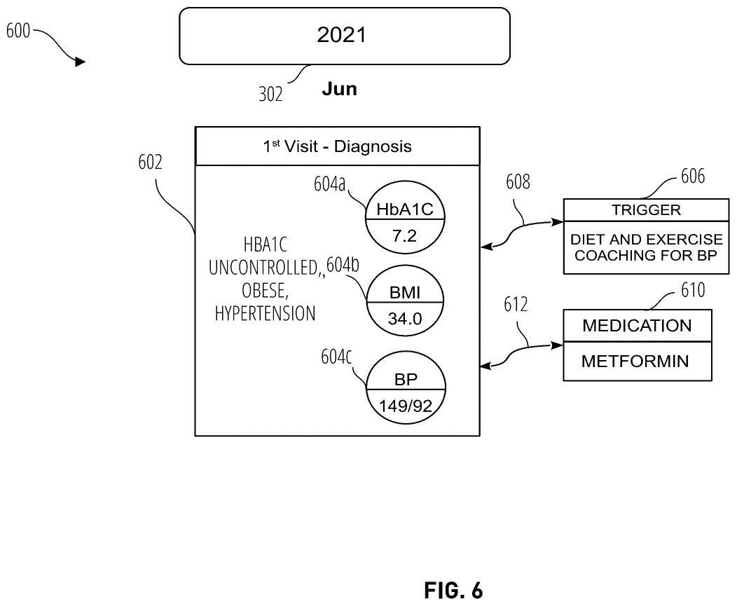

While the process 200 is described in conjunction with a patient treatment history that includes multiple patient visits for illustrative purposes, the CDS system 102 and the process 200 also generate a user interface with a simplified timeline view for an initial patient visit in which only a single patient visit is depicted in the timeline view using the same processing steps that are described above. FIG. 6 depicts a timeline view 600 of an initial patient visit for a PwD patient who is either diagnosed with a form of diabetes or is considered at-risk for diabetes. The timeline view 600 includes a graphical element 602 with an indication of a diagnosis for the PwD as having uncontrolled HbAlc, being obese, and having hypertension. The graphical element 602 also includes graphical sub-elements 604a indicating the HbAlc physiological parameter, 604b indicating the BMI physiological parameter, and 604c indicating the blood pressure (BP) physiological parameters that are relevant to the diagnoses for the PwD. The timeline view 600 further includes graphical elements 606 and 610 that correspond to recommended prescribed treatment triggers for diet and exercises coaching to address the elevated blood pressure (graphical element 606) and a recommended prescribed medication for metformin (graphical element 610). Graphical connectors 608 and 612 link the graphical element 602 for the initial patient visit to the graphical elements 606 and 610, respectively, for the recommended prescribed treatments. The timeline view 600 also includes a simplified timeline element 302 that indicates the year and month date of the initial patient visit, but that omits a timeline slider control because the patient visit history only includes the initial visit.

The embodiments described herein enable the generation of timeline view user interfaces that present highly relevant diagnosis, physiological parameter, and recommended prescribed treatment options to an HCP. The user interface reduces the cognitive burden on the HCP to enable more efficient and effective treatment for PwDs and other patients. When integrated within a clinical workflow, the embodiments described herein can help HCPs in making better personalized therapy decisions improving clinical, patient-reported and economical outcomes. In particular, the embodiments described herein enable an HCP to work with existing clinical guidelines for therapy transitions in diabetes in an efficient manner, because these guidelines may be cumbersome to follow or apply given the volume of patient data in prior-art systems. In addition, the embodiments described herein simplify the HCP's analysis of potential courses of recommended therapy that can be complex and evolve over several years, which simplifies the review of a PwD's treatment history and tracking of disease progression with an adaptive user interface that is customized for the specific physiological parameters, diagnoses, and prescribed treatments for each PwD.

This disclosure is described in connection with what are considered to be the most practical and preferred embodiments. However, these descriptions are presented by way of illustration and are not intended to be limited to the disclosed embodiments. Accordingly, one of skill in the art will realize that this disclosure encompasses all modifications and alternative arrangements within the spirit and scope of the disclosure and as set forth in the following claims.

What is claimed is:

1. A method for generating a user interface of a treatment history for a patient comprising:
receiving, with a processor, medical data for the patient, the medical data corresponding to a plurality of patient visits to a healthcare provider; and
generating, with the processor, graphical data corresponding to a timeline view of the plurality of patient visits, the generating of the graphical data further comprising:
generating a first graphical element corresponding to a first diagnosis based on the medical data during a current patient visit in the plurality of patient visits, the first graphical element further comprising:
a graphical indicator of the first diagnosis for a first medical condition; and
at least one graphical sub-element, the at least one graphical sub-element being relevant to a physiological parameter selected from the medical data, the physiological parameter being related to the first diagnosis; and
generating a second graphical element corresponding to a second diagnosis based on the medical data for the patient during a first prior patient visit in the plurality of patient visits, the second graphical element further comprising:
a graphical indicator of the second diagnosis for a second medical condition; and
at least one graphical sub-element, the at least one graphical sub-element being relevant to a physiological parameter selected from the medical data, the physiological parameter being related to the second diagnosis; and
generating a first graphical connector between the second graphical element and the first graphical element, the first graphical connector indicating a progression of time between the first prior patient visit and the current patient visit in the timeline view.

2. The method of claim 1, the generating, with the processor, of the graphical data corresponding to the timeline view further comprising:
generating graphical data corresponding to a timeline slider in the timeline view;
generating a third graphical element relevant to a third diagnosis in the medical data during a second prior patient visit in the plurality of patient visits, the second prior patient visit occurring prior to the first prior patient visit, in response to a user input to the timeline slider that expands a time range depicted in the timeline view, the third graphical element further comprising:
a graphical indicator of the third diagnosis for a third medical condition; and
at least one graphical sub-element, the at least one graphical sub-element being relevant to a physiological parameter selected from the medical data, the physiological parameter being related to the third diagnosis; and
generating a second graphical connector between the third graphical element and the second graphical element, the graphical connector indicating a progression of time between the second prior patient visit and the first prior patient visit in the timeline view.

3. The method of claim 1, the generating, with the processor, of the graphical data corresponding to the timeline view further comprising:
generating graphical data corresponding to a timeline slider in the timeline view;
generating a third graphical element relevant to a third diagnosis in the medical data during a second prior patient visit in the plurality of patient visits, the second prior patient visit occurring prior to the first prior patient visit, in response to a user input to the timeline slider that moves to an earlier time range in the timeline view, the third graphical element further comprising:
a graphical indicator of the third diagnosis for a third medical condition; and
at least one graphical sub-element, the at least one graphical sub-element being relevant to a physiological parameter selected from the medical data, the physiological parameter being related to the third diagnosis;
generating a second graphical connector between the second graphical element and the third graphical element, the graphical connector indicating a progression of time between the second prior patient visit and the first prior patient visit in the timeline view; and
removing the first graphical element from the timeline view in response to the current patient visit occurring outside of the earlier time range in the timeline view.

4. The method of claim 1, the generating, with the processor, of the graphical data corresponding to the timeline view further comprising:
generating a third graphical element of a timeline that indicates a first date of the first prior patient visit and a second date of the current patient visit.

5. The method of claim 1, the generating, with the processor, of the graphical data corresponding to the timeline view further comprising:
generating a third graphical element including a graphical indicator of a prescribed treatment that is prescribed during the first prior patient visit, wherein the first graphical connector further comprises a first sub-connector that connects the second graphical element of the first prior patient visit to the third graphical element and a second sub-connector that connects the third graphical element to the first graphical element of the current patient visit to indicate that the patient receives the prescribed treatment between the first prior patient visit and the current patient visit in the timeline view.

6. The method of claim 5, the generating, with the processor, of the third graphical element further comprising:
generating the third graphical element including a graphical indicator identifying that the prescribed treatment was manually selected by a healthcare provider.

7. The method of claim 5, the generating, with the processor, of the third graphical element further comprising:
generating the third graphical element including a graphical sub-element corresponding to a clinical note associated with the prescribed treatment.

8. The method of claim 1, the generating, with the processor, of the graphical data corresponding to the timeline view further comprising:
generating a third graphical element including a graphical indicator of a recommendation for a prescribed treatment that is relevant to the first diagnosis;
generating a second graphical connector between the first graphical element and the third graphical element, the second graphical connector indicating that the recommendation for the prescribed treatment is relevant to the first diagnosis.

9. The method of claim 1 further comprising:
generating the graphical data corresponding to the timeline view with the processor being provided in a server computing system; and
transmitting, with the processor and a network transceiver, the graphical data corresponding to the timeline view to a client computing system for display with a display device provided in the client computing system.

10. The method of claim 1 further comprising:
generating the graphical data corresponding to the timeline view with the processor being provided in a computing system; and
displaying the graphical data corresponding to the timeline view with a display device provided in the computing system.

11. The method of claim 10, wherein the computing system is a terminal of a healthcare provider.

12. A computing system configured to generate a user interface of a treatment history for a patient comprising:
a memory configured to store:
medical data for the patient, the medical data corresponding to a plurality of patient visits to a healthcare provider; and
stored program instructions; and
a processor operatively connected to the memory, the processor being configured to execute the stored program instructions to:
generate graphical data corresponding to a timeline view, the graphical data further comprising:
a first graphical element corresponding to a first diagnosis based on the medical data during a current patient visit in the plurality of patient visits, the first graphical element further comprising:
a graphical indicator of the first diagnosis for a first medical condition; and
at least one graphical sub-element, the at least one graphical sub-element being relevant to a physiological parameter selected from the medical data, the physiological parameter being related to the first diagnosis;
a second graphical element corresponding to a second diagnosis based on the medical data for the patient during a first prior patient visit in the plurality of patient visits, the second graphical element further comprising:
a graphical indicator of the second diagnosis for a second medical condition; and
at least one graphical sub-element, the at least one graphical sub-element being relevant to a physiological parameter selected from the medical data, the physiological parameter being related to the second diagnosis; and
a first graphical connector between the second graphical element and the first graphical element, the first graphical connector indicating a progression of time between the first prior patient visit and the current patient visit in the timeline view.

13. The computing system of claim 12, the processor being further configured to:
generate graphical data corresponding to a timeline slider in the timeline view;
generate a third graphical element relevant to a third diagnosis in the medical data during a second prior patient visit in the plurality of patient visits, the second prior patient visit occurring prior to the first prior patient visit, in response to a user input to the timeline slider that expands a time range depicted in the timeline view, the third graphical element further comprising:
a graphical indicator of the third diagnosis for a third medical condition;
at least one graphical sub-element, the at least one graphical sub-element being relevant to a physiological parameter selected from the medical data, the physiological parameter being related to the third diagnosis; and
a graphical indicator of a third prescribed treatment for the patient based on the third diagnosis; and
generate a second graphical connector between the second graphical element and the third graphical element, the graphical connector indicating a progression of time between the second prior patient visit and the first prior patient visit in the timeline view.

14. The computing system of claim 12, the processor being further configured to:
generate graphical data corresponding to a timeline slider in the timeline view;
generate a third graphical element relevant to a third diagnosis in the medical data during a second prior patient visit in the plurality of patient visits, the second prior patient visit occurring prior to the first prior patient visit, in response to a user input to the timeline slider that moves to an earlier time range in the timeline view, the third graphical element further comprising:
a graphical indicator of the third diagnosis for a third medical condition; and
at least one graphical sub-element, the at least one graphical sub-element being relevant to a physiological parameter selected from the medical data, the physiological parameter being related to the third diagnosis;
generate a second graphical connector between the second graphical element and the third graphical element, the graphical connector indicating a progression of time between the second prior patient visit and the first prior patient visit in the timeline view; and
remove the first graphical element from the timeline view in response to the current patient visit occurring outside of the earlier time range in the timeline view.

15. The computing system of claim 12, the processor being further configured to:
generate a third graphical element of a timeline that indicates a first date of the first prior patient visit and a second date of the current patient visit.

16. The computing system of claim 12, the processor being further configured to:
generate a third graphical element including a graphical indicator of a prescribed treatment that is prescribed during the first prior patient visit, wherein the first graphical connector further comprises a first sub-connector that connects the second graphical element of the first prior patient visit to the third graphical element and a second sub-connector that connects the third graphical element to the first graphical element of the current patient visit to indicate that the patient receives the prescribed treatment between the first prior patient visit and the current patient visit in the timeline view.

17. The computing system of claim 16, the processor being further configured to:
generate the third graphical element including a graphical indicator identifying that the prescribed treatment was manually selected by a healthcare provider.

18. The computing system of claim 16, the processor being further configured to:
generate the third graphical element including a graphical sub-element corresponding to a clinical note associated with the prescribed treatment.

19. The computing system of claim 12, the processor being further configured to:
generate a third graphical element including a graphical indicator of a recommendation for a prescribed treatment that is relevant to the first diagnosis;
generate a second graphical connector between the first graphical element and the third graphical element, the second graphical connector indicating that the recommendation for the prescribed treatment is relevant to the first diagnosis.

20. The computing system of claim 12 further comprising:
a network transceiver; and
the processor being operatively connected to the network transceiver and further configured to:
generate the graphical data corresponding to the timeline view with the processor being provided in a server computing system; and
transmit, with the network transceiver, the graphical data corresponding to the timeline view to a client computing system for display with a display device provided in the client computing system.

21. The computing system of claim 12 further comprising:
a display device; and
the processor being operatively connected to the display device and further configured to:
display the graphical data corresponding to the timeline view with the display device.

22. The computing system of claim 21, wherein the computing system is a terminal of a healthcare provider.

23. A method for generating a user interface of a treatment history for a patient comprising:
receiving, with a processor, medical data for the patient, the medical data corresponding to an initial patient visit to a healthcare provider; and
generating, with the processor, graphical data corresponding to a timeline view, the generating of the graphical data further comprising:
generating a first graphical element corresponding to a first diagnosis based on the medical data during the initial patient visit, the first graphical element further comprising:
a graphical indicator of the first diagnosis for a first medical condition; and
at least one graphical sub-element, the at least one graphical sub-element being relevant to a physiological parameter selected from the medical data, the physiological parameter being related to the first diagnosis.

24. The method of claim 23 the generating, with the processor, of the graphical data corresponding to the timeline view further comprising:
generating a second graphical element including a graphical indicator of a recommendation for a prescribed treatment that is relevant to the first prescribed diagnosis;
generating a graphical connector between the first graphical element and the second graphical element, the graphical connector indicating that the recommendation for the prescribed treatment is relevant to the first diagnosis.

25. The method of claim 23, the generating, with the processor, of the graphical data corresponding to the timeline view further comprising:
generating a second graphical element of a timeline that indicates a date of the initial patient visit.

26. The method of claim 23 further comprising:
generating the graphical data corresponding to the timeline view with the processor being provided in a server computing system; and
transmitting, with the processor and a network transceiver, the graphical data corresponding to the timeline view to a client computing system for display with a display device provided in the client computing system.

27. The method of claim 23 further comprising:
generating the graphical data corresponding to the timeline view with the processor being provided in a computing system; and
displaying the graphical data corresponding to the timeline view with a display device provided in the computing system.

28. The method of claim 27, wherein the computing system is a terminal of a healthcare provider.
